(19) United States
(12) Patent Application Publication
(10) Pub. No.: US 2024/0299723 A1
(43) Pub. Date: Sep. 12, 2024

(54) MALE LUER CONNECTOR

(71) Applicant: CareFusion 303, Inc., San Diego, CA (US)

(72) Inventors: Yan FANG, Irvine, CA (US); Aaron En-Yu WANG, Laguna Hills, CA (US)

(21) Appl. No.: 18/179,656

(22) Filed: Mar. 7, 2023

(51) Int. Cl. A61M 39/10

(52) U.S. Cl. CPC A61M 39/10; A61M 2039/1027; A61M 2039/1083; A61M 2205/02

(57) ABSTRACT

A male luer connector is described herein. The luer connector can include a luer slip, a collar, and a spigot. The luer slip can include a first end and a second end and define a lumen extending between the first end and the second end. The first end of the luer slip can be configured to engage with a mating connector. The collar can be disposed around the luer slip. The collar can be translatable relative the luer slip. The collar is configured to couple with the mating connector adjacent to the first end of the luer slip. The spigot defines a fluid port. The spigot is coupled to the luer slip and the fluid port is in fluid communication with the lumen at the second end of the luer slip.

### TECHNICAL FIELD

The present disclosure generally relates to infusion set intravenous (IV) connectors, in particular male luer connectors.

### BACKGROUND

Typical infusion or intravenous (IV) sets are constructed by joining polymeric tubing segments to polymeric components, many of which use luer connectors. These IV sets can be used with infusion pumps or gravity systems to provide fluids to a user, such as a patient. However, typical luer connectors generally achieve an optimized key function by sacrificing another function. For instance, in order to have a better gripping feature, typical luer connectors tend to be bulky and/or have sharp features, which leads to higher costs and negatively affect patient comfort. In some applications, typical luer connectors can be formed from acrylonitrile butadiene styrene (ABS), polyvinyl chloride (PVC), or acrylic.

### SUMMARY

Luer connectors made from acrylonitrile butadiene styrene (ABS), polyvinyl chloride (PVC), acrylic, or other similar materials may degrade after chemical exposure. Degraded luer connectors may by more prone to cracking and/or breaking. In some applications, degraded luer connectors may be more prone to inadvertently chemically bonding, preventing disconnection of the luer connector.

For these reasons, it is desirable to provide male luer connectors for use with IV components and IV sets that optimize important aspects of a male luer connector (e.g., connection, disconnection, cleaning and comfort) with one integrated design. Further, it may be desirable to provide male luer connectors for use with IV components and IV sets that resist degradation from chemical exposure.

In accordance with at least some embodiments disclosed herein is the realization that certain conventional connectors may be difficult to grip, manipulate, or otherwise operate with a single hand or in a single motion, difficult to disconnect, and/or difficult to clean which may result in clinicians requiring additional time to connect and disconnect fluid connections. Further, in accordance with at least some embodiments disclosed herein is the realization that certain conventional connectors may be bulky, which may result in patient discomfort and inadvertent disconnections. Further, in accordance with at least some embodiments disclosed herein is the realization that certain conventional connectors may be formed of materials that may degrade upon exposure to chemicals such as medical fluids and/or solvents, which may result in cracking, leaking, and/or contamination of medical fluids.

Accordingly, aspects of the present disclosure provide a connector comprising: a first connector portion comprising: a luer slip comprising a first end and a second end, and defining a lumen extending between the first end and the second end, wherein the first end is configured to engage with a mating connector; a collar disposed around the luer slip, wherein the collar is translatable relative to the luer slip and the collar is configured to couple with the mating connector adjacent to the first end of the luer slip; and a spigot defining a fluid port, wherein the spigot is coupled to the luer slip and the fluid port is in fluid communication with the lumen at the second end of the luer slip.

In some instances, the present disclosure provides an infusion set comprising an infusion set component; tubing coupled to the infusion set component; and a male luer connector, comprising: a luer slip comprising a first end and a second end, and defining a lumen between the first end and the second end, wherein the first end is configured to engage with a mating connector, and the luer slip is coupled to the tubing.

In some instances, the present disclosure provides a connector assembly comprising a female luer connector, comprising: a receiving portion; a first fluid port configured to receive intravenous tubing; and a female luer connector lumen fluidly coupled between the receiving portion and the first fluid port; and a male luer connector, comprising: a luer slip comprising a first end and a second end, and a plurality of protrusions extending radially from a slip surface, and the luer slip defining a lumen extending between the first end and the second end, wherein the first end is configured to engage with a mating connector; a collar disposed around the luer slip and defining a plurality of notches corresponding to the plurality of protrusions of the luer slip, wherein the collar is translatable relative to the luer slip, the collar is configured to couple with the mating connector adjacent to the first end of the luer slip, and the plurality of notches are configured to engage with the plurality of protrusions when the collar is spaced apart from the first end of the luer slip, selectively rotationally coupling the collar and the luer slip; and a spigot comprising a teardrop shape and defining a fluid port, wherein the spigot is coupled to the luer slip and the fluid port is in fluid communication with the lumen at the second end of the luer slip, and the spigot defines at least one gripping feature.

Accordingly, the present application addresses several operational challenges encountered in prior fluid connections and provides numerous improvements that enable the user to increase safety and efficacy, while more easily providing fluid connections.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIGS. 2A-2C are top views of a typical assembled IV infusion set and typical IV extension lines.

FIG. 3 depicts a side perspective view of a male luer connector, according to aspects of the disclosure.

FIG. 4 depicts a front perspective view of the male luer connector of FIG. 3, according to aspects of the disclosure.

FIG. 5 depicts a perspective view of a luer connector assembly, according to aspects of the disclosure.

FIG. 6 depicts a perspective view of a male luer connector coupled to an IV tube beside a typical male luer connector, according to aspects of the disclosure.

FIG. 7 depicts a side elevation view of a male luer connector coupled to a micro bore or small bore tubing, according to aspects of the disclosure.

FIG. 8 depicts a side elevation view of a male luer connector coupled to a micro bore or small bore tubing, according to aspects of the disclosure.

FIG. 9 depicts a side elevation view of a male luer connector coupled to a standard bore tubing, according to aspects of the disclosure.

FIG. 10 depicts a side elevation view of a male luer connector coupled to a macro bore tubing, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

In accordance with some embodiments, the present application discloses various features and advantages of a fluid connector. The connectors described herein can provide features that allow for a clinician to easily grasp, manipulate, connect, and disconnect fluid connections during operation, avoiding the use of additional tools to manipulate the connectors. Further, the connectors described herein can include features to allow for easier cleaning. In some applications, the connectors can have a smaller profile or overall dimensions, and can be formed without any sharp edges, increasing patient comfort. Further, certain connectors can be formed from materials that resist degradation from chemical exposure, preventing cracking, leaking, and inadvertent bonding. In some applications, certain connectors can be coupled to tubing mechanically without the use of adhesives. In some applications, certain connectors can be configured to be used with tubing of various sizes, minimizing the number of different connector types required to be manufactured and stored in inventory.

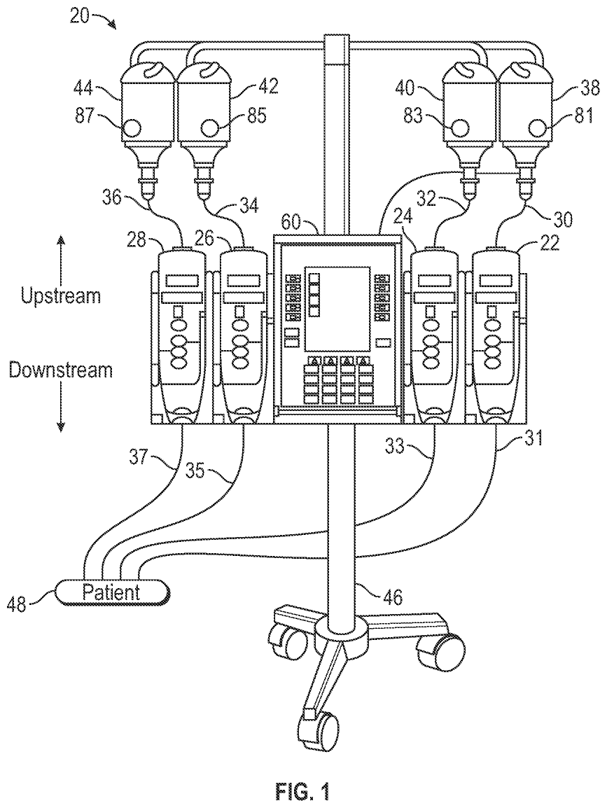

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

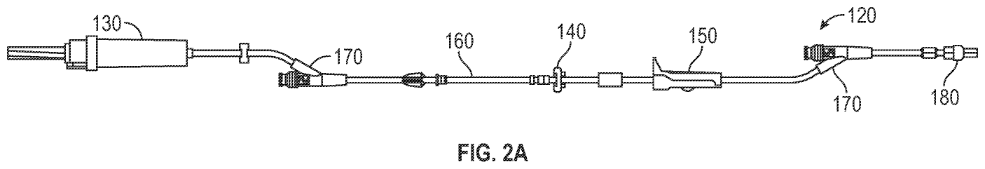

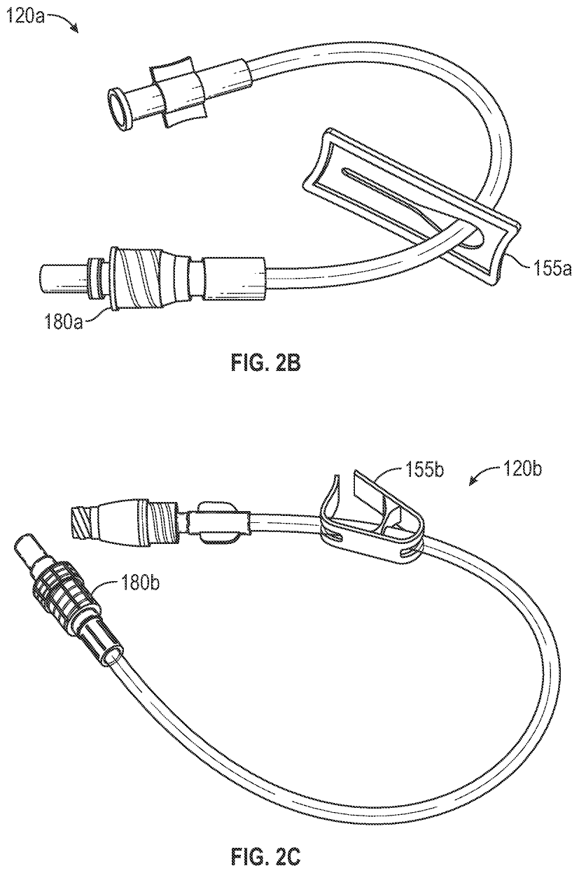

Typically, medical fluid administration sets have more parts than are shown in FIG. 1, such as those shown in FIGS. 2A-2C. Infusion sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIGS. 2A, an infusion set 120 may include a drip chamber 130, a check valve 140 and a roller clamp 150 connected together by tubing 160. The infusion set 120 may also include a Y-site 170 having a Y-shaped junction with a needleless connector 175, as well as a luer lock connector 180 at the end of the infusion set 120. The luer lock connector 180 may be used for connection to a catheter inserted into a patient or connecting to any other suitable tubing or component. The infusion set 120 may include additional infusion components and may be formed of any combination of components and the tubing 160.

As shown in FIG. 2B, an IV extension set 120a may be used to connect any two infusion components or devices, such as IV sets, infusion pumps, syringe pumps and the like. IV extension set 120a includes a luer connector 180a and a flow controller 155a. Similarly, as shown in FIG. 2C, another IV extension set 120b includes a luer connector 180b and a flow controller 155b.

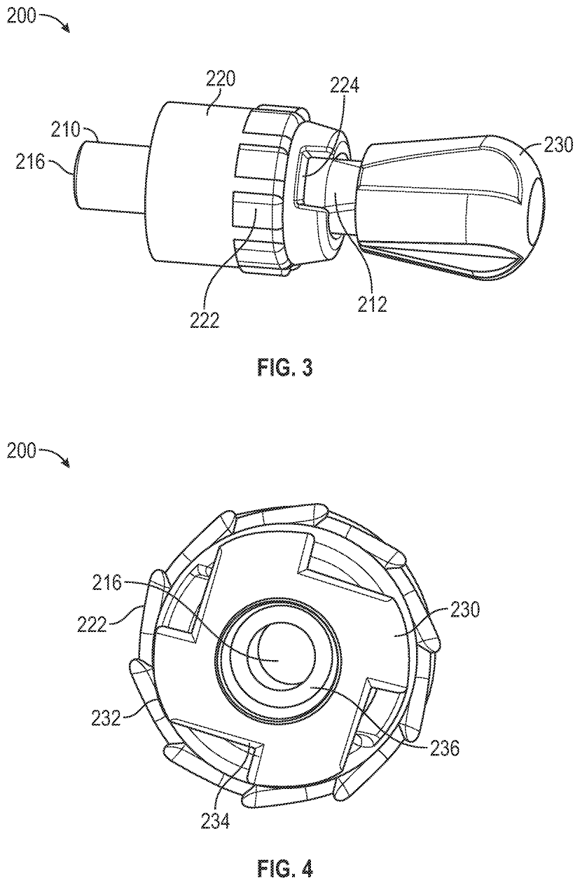

With reference to FIGS. 3 and 4, a male luer connector 200 is provided according to aspects of the disclosure. In the depicted example, the male luer connector 200 facilitates a physical connection and fluid flow between tubing and/or components of an IV set. In the depicted example, the male luer connector 200 includes a luer slip 210 that defines a flow path between the ends of the male luer connector 200. As illustrated, the luer slip 210 includes a hollow body that defines a lumen 216 along the length of the luer slip 210.

As described herein, the male luer connector 200 and components thereof can be formed from chemical resistant materials. Advantageously, by forming the components of the male luer connector 200 from chemical resistant materials, the male luer connector 200 may resist degradation after being exposed to medical fluids and/or other chemicals. In the depicted example, the luer slip 210 and/or other components of the male luer connector 200 can be formed from a chemical resistant material. In some applications the luer slip 210 is formed from polypropylene. Advantageously, certain polypropylene resins may be more cost effective than acrylonitrile butadiene styrene. In some applications, the luer slip 210 can be formed from other suitable materials, including, but not limited to, polycarbonate, nylon, or polyethylene.

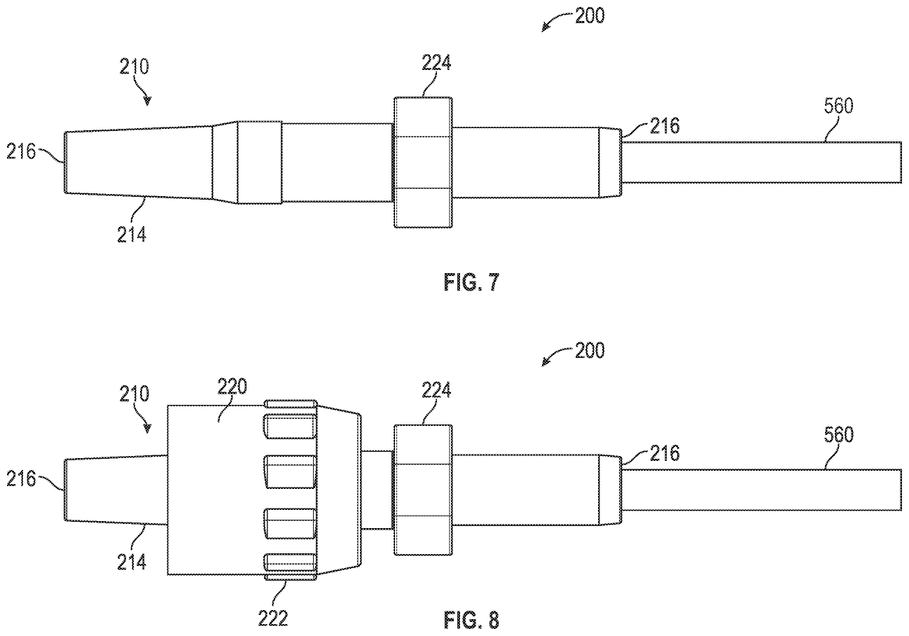

In the depicted example, the male luer connector 200 couples to tubing to terminate the end of the tubing and facilitate fluid connections. As described herein (and with reference to FIGS. 7-10), the male luer connector 200 can be utilized in one or more configurations to allow to allow tubing of various sizes or types to coupled to the male luer connector 200. For example, the male luer connector 200 can be adapted, adjusted, or otherwise configured to be used with at least the following types of tubing:
(i) Micro Bore (T0.5×2.3)—tubing OD: approximately 2.30 mm/0.091 in; tubing ID: approximately 0.50 mm/0.020 in; wall thickness: approximately 0.90 mm/0.035 in
(ii) Small Bore (T1×2.3)—tubing OD: approximately 2.30 mm/0.091 in; tubing ID: approximately 1.00 mm/0.039 in; wall thickness: approximately 0.65 mm/0.026 in
(iii) Standard Bore Thick Wall (T1.5×3.7)—tubing OD: approximately 3.70 mm/0.146 in; tubing ID: approximately 1.50 mm/0.059 in; wall thickness: approximately 1.10 mm/0.043 in
(iv) Standard Bore (T2.7×3.7)—tubing OD: 3.70 mm/0.146 in; tubing ID: approximately 2.70 mm/0.106 in; wall thickness: approximately 0.50 mm/0.020 in
(v) Macro Bore (T3×4.2)—tubing OD: approximately 4.20 mm/0.165 in; tubing ID approximately 3.00 mm/0.118 in; wall thickness: approximately 0.60 mm/0.024 in

In some applications, the measurements of the tubing can have the following tolerances: tubing OD: approximately 0.08 mm/0.003 in; tubing ID: approximately 0.05 mm/0.002 in; wall thickness: approximately 0.09 mm/0.004 in. Advantageously, by allowing various types or sizes of tubing (e.g., micro bore, small bore, standard bore, and macro bore tubing) to couple with the male luer connector 200, a single male luer connector 200 design can be utilized with multiple tubing types or sizes, simplifying the number of connector types needed for IV set connections, reducing inventories, and simplifying manufacturing.

In some applications, the male luer connector 200 can be configured to couple the tubing directly to the luer slip 210 or configured to couple the tubing to a spigot 230, which is in turn coupled to the luer slip 210. Advantageously, the multiple configurations of the male luer connector 200 allow tubing of various sizes to the coupled to the male luer connector 200.

In some configurations, the tubing is coupled directly to an end of the luer slip 210. The tubing can be received within the lumen 216 of the luer slip 210. Optionally, the luer slip 210 can define a pocket within the lumen 216 or concentric with the lumen 216 to receive the tubing. In some applications, the tubing can be adhesively bonded within the luer slip 210. The adhesive can be UV cured or activated. In some applications, a micro bore tubing or a small bore tubing can be coupled to the luer slip 210.

In some applications, the tubing is coupled to a spigot 230, which in turn, is coupled to the luer slip 210 of the male luer connector 200. Optionally, the tubing can be received within a fluid port 236 of the spigot 230. The tubing can be mechanically coupled to the spigot 230. Optionally, the tubing can be captured between the luer slip 210 and the spigot 230 to mechanically couple the spigot 230. In some applications, the mechanical coupling of the tubing with the spigot 230 eliminates the need for adhesives or any other bonding agent. Advantageously, the use of a mechanical coupling can minimize solvent exposure and environmental footprint of the connector.

In the depicted example, the spigot 230 is coupled to the luer slip 210. In some applications, the spigot 230 is circumferentially disposed around the luer slip 210. The spigot 230 can be separately formed from the luer slip 210 or integrally formed with the luer slip 210 (e.g., via molding). In some applications, the spigot 230 can be sealed against the luer slip 210 to minimize leaking. Advantageously, the arrangement of the male luer connector 200 and the use of mechanical coupling can simplify the design of the junction, reducing the risk of leaking, separation, and/or occlusion. Further, the assembly of the junction may be readily automated. In some applications a standard bore tubing or a macro bore tubing can be coupled to the male luer connector 200 via the spigot 230 coupled to the luer slip 210.

In the depicted example, the spigot 230 can be formed from a chemical resistant material. In some applications the spigot 230 is formed from polypropylene. Advantageously, mechanical coupling of tubing can be utilized with materials that may not readily bond with solvent, such as polypropylene. In some applications, the spigot 230 can be formed from other suitable materials, including, but not limited to, polycarbonate, nylon, or polyethylene.

Further, in some applications, the spigot 230 may be formed of a material that is soft and cushioned, such as silicone, polyurethane, a latex free silicone rubber, and the like, or any combination thereof. In this case, material of the spigot 230 may be overmolded or otherwise adhered to the luer slip 210 and the soft cushioning property of the spigot 230 may allow the male luer connector 200 to be pressed against a patient's skin for a prolonged period of time without causing skin tears or pressure sores. The spigot 230 may be latex free, providing for use in a wide range of medical applications, including those that proscribe latex contact with the patient's skin (e.g., patient has latex allergy). The spigot 230 may be formed to withstand gas sterilization and gamma sterilization.

In the depicted example, the male luer connector 200 can engage with a mating a standard female receptacle. For example, the male luer connector 200 can mate with a connector of an infusion set device or component (e.g., female luer connector, female needle free port, female catheter set, female pump port) that is configured to connect with a male luer connector. In the depicted example, the opposite end of the luer slip 210 can configured to engage with a mating female luer connector.

In the depicted example, a collar 220 couples and secures the male luer connector 200 with the mating connector after the luer slip 210 has engaged the mating connector. In some applications, the collar 220 can be rotated to threadedly engage mating threads of the mating connector to couple the male luer connector 200 with the mating female connector. Optionally, the collar 220 can use any other suitable engagement mechanism to couple the male luer connector 200 with a mating connector.

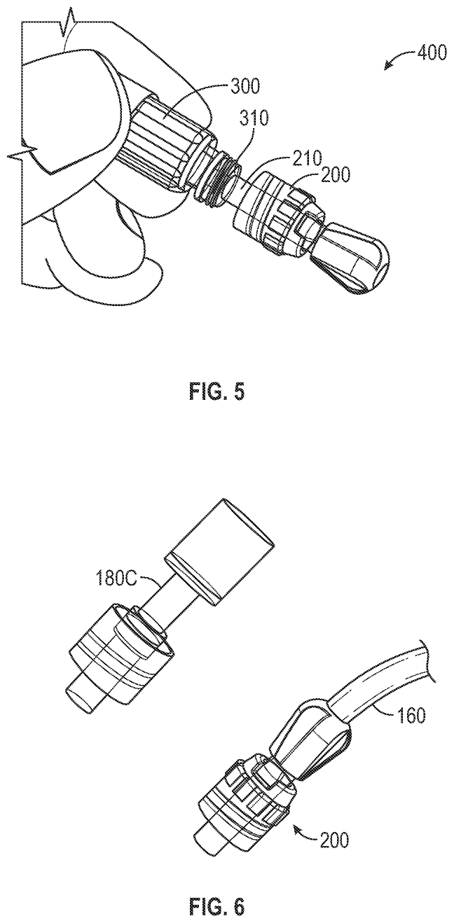

As illustrated, the collar 220 is disposed around and coupled to the luer slip 210. In some embodiments, the collar 220 may be slidingly coupled relative to the luer slip 210, permitting the collar 220 to slide relative to the luer slip 210. Optionally, the sliding travel of the collar 220 may be limited or configured to enable the luer slip 210 to be fully engaged with a female connector (e.g., female luer connector 300 shown in FIG. 5) before the collar 220 contacts the female luer connector 300, while providing the ability to maintain a firm grip on the male luer connector 200. Advantageously, the sliding travel of the collar 220 can be selected to ensure a leakage free connection between the male luer connector 200 and the female luer connector 300. Further, the sliding travel of the collar 220 can be configured to give the user (e.g., healthcare provider, clinician) more space to clean the luer slip 210. Optionally, the sliding travel of the collar 220 can be configured or limited for any suitable purpose.

In the depicted example, the collar 220 can be formed from a chemical resistant material. In some applications the collar 220 is formed from polypropylene. In some applications, the collar 220 can be formed from other suitable materials, including, but not limited to, polycarbonate, nylon, or polyethylene.

As another example, the collar 220 may be formed of a material that is soft and cushioned, such as silicone, polyurethane, a latex free silicone rubber, and the like, or any combination thereof. In this case, the soft cushioning property of the collar 220 may allow the male luer connector 200 to be pressed against a patient's skin for a prolonged period of time without causing skin tears or pressure sores. The collar 220 may be latex free, providing for use in a wide range of medical applications, including those that proscribe latex contact with the patient's skin (e.g., patient has latex allergy). The collar 220 may be slip resistant when wet and may be formed to hold up to gas sterilization and gamma sterilization.

The collar 220 may be textured and/or contoured to provide for an improved grip when the male luer connector 200 is to be loosened or tightened. For example, as shown in FIGS. 3 and 4, the collar 220 may have multiple raised features 222, such as the angled projections with rounded leading edges as shown, which may provide texturing for non-slip and/or easy grip properties (e.g., grip features). The raised features 222 may be any suitable feature, such as ridges or other raised areas, for example.

In some embodiments, the male luer connector 200 includes features to retain the collar 220 in an engaged position relative to the luer slip 210 to facilitate the disconnection and/or removal of the male luer connector 200 from a mating female luer connector. As illustrated, the luer slip 210 can include one or more protrusions 212 extending radially from the surface of the luer slip 210 to retain the collar 220 in a desired engaged or coupled position with the luer slip 210. In some embodiments, the one or more protrusions 212 can engage with the collar 220 to retain the collar 220 in a desired rotational position relative to the luer slip 210 to prevent inadvertent slipping or rotation of the collar 220 relative to the luer slip 210.

In the depicted example, the collar 220 includes or more notches 224 to selectively engage the luer slip 210 and prevent rotation of the collar 220 relative to the luer slip 210. As illustrated, the notches 224 can be circumferential recesses in a trailing edge of the collar 220. Upon disconnecting the collar 220 from a mating connector, the collar 220 can be slid distally or back along its sliding travel towards the protrusions 212 to allow the notches 224 of the collar 220 to engage the protrusions 212 of the luer slip 210. In some embodiments, the collar 220 can be rotated to engage the notches 224 with the protrusions 212 of the luer slip 210 and prevent further rotation of the collar 220 relative to the luer slip 210 during disconnection. Further, the notches 224 may be slanted or angled to facilitate rotational alignment and engagement with the protrusions 212 of the luer slip 210 to increase biting or engagement force and/or avoid slipping or rotation of the collar 220 relative to the luer slip 210 while being disconnected from the mating connector. Optionally, the notches 224 and/or the protrusions 212 can sized, shaped, or otherwise configured to provide tactile or auditory feedback of engagement, such as a “click” to provide confirmation of engagement. Advantageously, the selective engagement of the collar 220 with the luer slip 210 can allow a clinician to disconnect the male luer connector 200 in a single motion, without allowing the collar 220 to rotate relative to the luer slip 210, increasing stability, and preventing the inadvertent or sudden ejection of components of the male luer connector 200 during disconnection.

As illustrated, the spigot 230 can be utilized as a grip for a clinician. In the depicted example, the spigot 230 can be sized and shaped to allow a clinician to readily and comfortably grasp, hold, and/or manipulate the male luer connector 200. In some embodiments, the spigot 230 can have a generally tapered tear drop shape. As illustrated, the spigot 230 can further include grip features 232 and recesses 234 to further allow a clinician to manipulate the spigot 230 and the male luer connector 200 generally. In some embodiments, the grip features 232 and the recesses 234 can be arranged in an angled “throwing star” arrangement.

In some applications, the shape and features of the spigot 230 can facilitate disconnection of the male luer connector 200 from a mating connector by allowing for a strong pulling force to be imparted by the clinician. Advantageously, the shape and features of the spigot 230 can allow for disconnection of the male luer connector 200 in applications where the luer slip 210 is stuck or otherwise requires high removal force (e.g., if disinfectant is utilized within the connection). Further, the shape and features of the spigot 230 allow for the profile of the spigot 230 to be disposed comfortably between a clinician's fingers, reducing or avoiding pressure points. Advantageously, the arrangement of the grip features 232 and the recesses 234 and the spigot 230 generally can allow for a slip-resistant grip when the male luer connector 200 is wet. Further, the arrangement of the spigot 230 can also reduce the potential for over-torquing the male luer connector 200.

As described herein, features of the male luer connector 200 can facilitate case of use compared to certain conventional connectors. In some applications, the features of the male luer connector 200, including, but not limited to, the sliding travel of the collar 220 relative to the luer slip 210, the selective engagement of the collar 220 with the luer slip 210, and/or the gripping features of the spigot 230 can allow for one hand placement and operation of the male luer connector 200. As described herein, the features described herein can allow for a clinician to rapidly and readily twist, pull, and otherwise manipulate the male luer connector 200 with a single hand. In some applications, the clinician may be able to use a single hand or finger placement to both twist and pull the male luer connector 200, allow for a rapid and efficient disconnection of the male luer connector 200.

FIG. 5 shows an example luer connector assembly 400, which includes the male luer connector 200 and a female luer connector 300. Here, the luer slip 210 of the male luer connector 200 engages or mates with a female port 310 of the female luer connector. For example, one of the male luer connector 200 and the female luer connector 300 may connect via IV tubing to an IV set (e.g., IV set 120) and the other of the male luer connector 200 and the female luer connector 300 may connect via IV tubing to a catheter inserted in a patient. Thus, the male luer connector 200 of the luer connector assembly 400 provides for easy one-handed connection to or disconnection from the female luer connector 300 for quick connection/disconnection of the IV set 120 and the catheter. As another example, a luer connector assembly 400 may be used between a fluid source (e.g., fluid supply 38) and a receiving end of the IV set 120 (e.g., drip chamber 130), thus providing for quick and easy connection/disconnection between the fluid supply 38 and the IV set 120.

As shown in FIG. 6, the male luer connector 200 may be compact with a minimized size in comparison to a typical male luer connector 180c. For example, the male luer connector 200 is about one half the volume of the typical male luer connector 180c, while the combination of the above-described features provides for stronger performance of the male luer connector 200 over the typical male luer connector 180c. Here, this size difference between the male luer connector 200 and the typical male luer connector 180c provides for reducing the material (e.g., resin) cost of the male luer connector 200 by half due to using half the volume of material. Also, the reduced size of the male luer connector 200 may be more comfortable on patients, which may be especially important for infants or babies in a neonatal intensive care unit (NICU) or a premature intensive care unit (PICU).

In aspects of the disclosure, male luer connector 200 may be formed in any suitable manner. For example, the luer slip 210 and the spigot 230 may be molded as an integral component, while the collar 220 may be molded as a separate component, after which the collar 220 may be slidable mounted onto the luer slip 210. As another example, the luer slip 210 and the spigot 230 may be separate components where the spigot 230 may be overmolded, welded or affixed by adhesive to the luer slip 210. The luer slip 210, the collar 220 and the spigot 230 may be formed from any combination of transparent materials, translucent materials and/or opaque materials.

FIG. 7 depicts a side elevation view of a male luer connector 200 coupled to a micro bore or small bore tubing 560, according to aspects of the disclosure. In the depicted example, the male luer connector 200 can be configured to allow tubing 560 to be coupled to the male luer connector 200. In some embodiments, the tubing 560 can be either micro bore tubing ((T0.5×2.3)—tubing OD: 2.30 mm/0.091 in; tubing ID 0.50 mm/0.020 in; wall thickness: 0.90 mm/0.035 in) or small bore tubing ((T1×2.3)—tubing OD: 2.30 mm/0.091 in; tubing ID 1.00 mm/0.039 in; wall thickness: 0.65 mm/0.026 in). In some applications, the tubing 560 can have an outer diameter of 2.30 mm/0.091 inches.

In some configurations, the tubing 560 is coupled directly to an end of the luer slip 210. As illustrated, the tubing 560 can be received within the lumen 216 of the luer slip 210. Optionally, the luer slip 210 can define a pocket within the lumen 216 or concentric with the lumen 216 to receive the tubing 560. In some applications, the tubing 560 can be adhesively bonded within the luer slip 210. The adhesive can be UV cured or activated.

In some applications, another portion of tubing can be coupled to the opposite end of the luer slip 210 to facilitate a fluid connection with the tubing 560. Tubing can be disposed over the outside diameter of the luer slip 210. The luer slip 210 can have a tapered or flared outer profile or diameter 214 to facilitate engagement of the tubing with the outer diameter of the luer slip 210. The tubing may deform to facilitate engagement or coupling with the luer slip 210.

FIG. 8 depicts a side elevation view of a male luer connector 200 coupled to a micro bore or small bore tubing 560, according to aspects of the disclosure. Similar to the configuration depicted in FIG. 7, the male luer connector 200 can be configured to allow micro bore or small bore tubing 560 to be coupled to the male luer connector 200.

In the configuration depicted in FIG. 8, the male luer connector 200 includes a collar 220 that couples and secures the male luer connector 200 with the mating connector to facilitate a fluid connection with the tubing 560. In some applications, the collar 220 can be rotated to threadedly engage mating threads of the mating connector to couple the male luer connector 200 with the mating female connector.

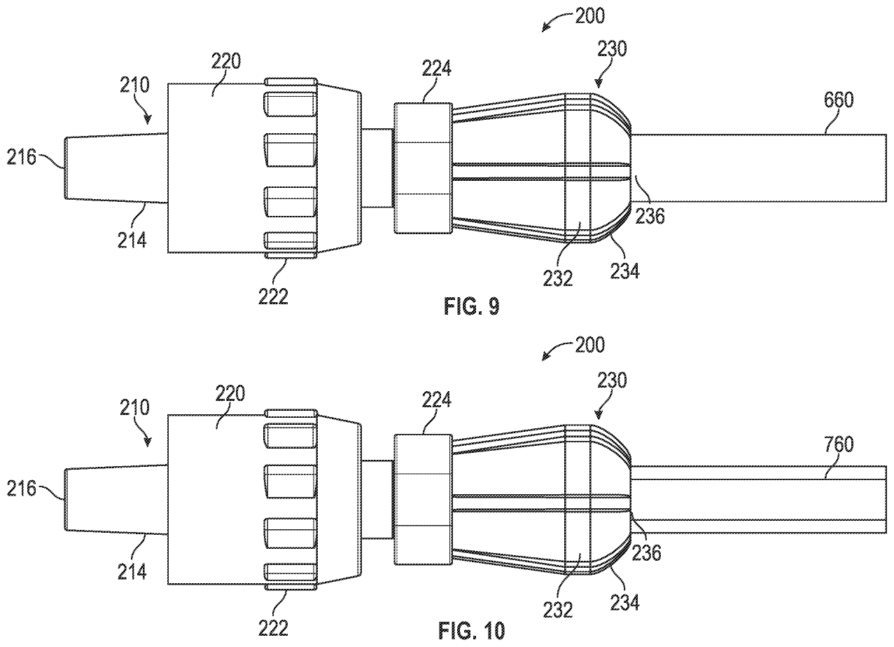

FIG. 9 depicts a side elevation view of a male luer connector 200 coupled to a standard bore tubing 660, according to aspects of the disclosure. In the depicted example, the male luer connector 200 can be configured to allow tubing 660 to be coupled to the male luer connector 200. In some embodiments, the tubing 660 can be either Standard Bore Thick Wall tubing ((T1.5×3.7)—tubing OD: 3.70 mm/0.146 in; tubing ID 1.50 mm/0.059 in; wall thickness: 1.10 mm/0.043 in) or Standard Bore tubing ((T2.7×3.7)—tubing OD: 3.70 mm/0.146 in; tubing ID 2.70 mm/0.106 in; wall thickness: 0.50 mm/0.020 in). In some applications, the tubing 660 can have an outer diameter of 3.70 mm/0.146 inches.

In some applications, the tubing 660 is coupled to a spigot 230, which in turn, is coupled to the luer slip 210 of the male luer connector 200. Optionally, the tubing 660 can be received within a fluid port 236 of the spigot 230. The tubing 660 can be mechanically coupled to the spigot 230. Optionally, the tubing 660 can be captured between the luer slip 210 and the spigot 230 to mechanically couple the spigot 230. In some applications, the mechanical coupling of the tubing 660 with the spigot 230 eliminates the need for adhesives or any other bonding agent. Advantageously, the use of a mechanical coupling can minimize solvent exposure and environmental footprint of the connector. As described herein, the spigot 230 can be coupled to the luer slip 210.

Similar to the configuration depicted in FIG. 8, in FIG. 9, the male luer connector 200 includes a collar 220 that couples and secures the male luer connector 200 with the mating connector to facilitate a fluid connection with the tubing 660.

FIG. 10 depicts a side elevation view of a male luer connector 200 coupled to a macro bore tubing 760, according to aspects of the disclosure. In the depicted example, the male luer connector 200 can be configured to allow tubing 760 to be coupled to the male luer connector 200. In some embodiments, the tubing 760 can be Macro Bore tubing ((T3×4.2)—tubing OD: 4.20 mm/0.165 in; tubing ID 3.00 mm/0.118 in; wall thickness: 0.60 mm/0.024 in).

In some applications, the tubing 760 is coupled to a spigot 230, which in turn, is coupled to the luer slip 210 of the male luer connector 200. Optionally, the tubing 760 can be received within a fluid port 236 of the spigot 230. The tubing 760 can be mechanically coupled to the spigot 230. Optionally, the tubing 760 can be captured between the luer slip 210 and the spigot 230 to mechanically couple the spigot 230.

Similar to the configuration depicted in FIG. 8, in FIG. 10, the male luer connector 200 includes a collar 220 that couples and secures the male luer connector 200 with the mating connector to facilitate a fluid connection with the tubing 760.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A male luer connector, comprising: a luer slip comprising a first end and a second end, and defining a lumen extending between the first end and the second end, wherein the first end is configured to engage with a mating connector; a collar disposed around the luer slip, wherein the collar is translatable relative to the luer slip and the collar is configured to couple with the mating connector adjacent to the first end of the luer slip; and a spigot defining a fluid port, wherein the spigot is coupled to the luer slip and the fluid port is in fluid communication with the lumen at the second end of the luer slip.

Clause 2. The male luer connector of Clause 1, wherein the second end of the luer slip is configured to receive tubing.

Clause 3. The male luer connector of Clause 2, wherein the lumen at the second end of the luer slip is configured to receive tubing.

Clause 4. The male luer connector of Clause 2, wherein the luer slip defines a pocket concentric to the lumen configured to receive tubing.

Clause 5. The male luer connector of Clause 2, wherein the tubing comprises micro bore tubing or small bore tubing.

Clause 6. The male luer connector of Clause 1, wherein the fluid port of the spigot is configured to receive tubing.

Clause 7. The male luer connector of Clause 6, wherein the tubing is disposed between the spigot and the luer slip.

Clause 8. The male luer connector of Clause 6, wherein the tubing comprises. standard bore thick wall tubing, standard bore tubing or macro bore tubing.

Clause 9. The male luer connector of Clause 1, wherein the luer slip is formed from polypropylene.

Clause 10. The male luer connector of Clause 1, wherein the collar is formed from polypropylene.

Clause 11. The male luer connector of Clause 1, wherein the spigot is formed from polypropylene.

Clause 12. An infusion set, comprising: an infusion set component; tubing coupled to the infusion set component; and a male luer connector, comprising: a luer slip comprising a first end and a second end, and defining a lumen between the first end and the second end, wherein the first end is configured to engage with a mating connector, and the luer slip is coupled to the tubing.

Clause 13. The infusion set of Clause 12, wherein the tubing is coupled to the second end of the luer slip.

Clause 14. The infusion set of Clause 12, wherein the tubing is received in a pocket defined by the luer slip, wherein the pocket is concentric to the lumen.

Clause 15. The infusion set of Clause 12, wherein the tubing comprises micro bore tubing or small bore tubing.

Clause 16. The infusion set of Clause 12, further comprising a spigot defining a fluid port, wherein the spigot is coupled to the luer slip, the fluid port is in fluid communication with the lumen at the second end of the luer slip, and the tubing is coupled to the fluid port.

Clause 17. The infusion set of Clause 16, wherein the tubing is disposed between the spigot and the luer slip.

Clause 18. The infusion set of Clause 16, wherein the tubing comprises standard bore thick wall tubing, standard bore tubing, or macro bore tubing.

Clause 19. The infusion set of Clause 12, further comprising a collar disposed around the luer slip, wherein the collar is translatable relative to the luer slip and the collar is configured to couple with the mating connector adjacent to the first end of the luer slip.

Clause 20. An infusion set luer connector assembly, comprising: a female luer connector, comprising: a receiving portion; a first fluid port configured to receive intravenous tubing; and a female luer connector lumen fluidly coupled between the receiving portion and the first fluid port; and a male luer connector, comprising: a luer slip comprising a first end and a second end, and a plurality of protrusions extending radially from a slip surface, and the luer slip defining a lumen extending between the first end and the second end, wherein the first end is configured to engage with a mating connector; a collar disposed around the luer slip and defining a plurality of notches corresponding to the plurality of protrusions of the luer slip, wherein the collar is translatable relative to the luer slip, the collar is configured to couple with the mating connector adjacent to the first end of the luer slip, and the plurality of notches are configured to engage with the plurality of protrusions when the collar is spaced apart from the first end of the luer slip, selectively rotationally coupling the collar and the luer slip; and a spigot comprising a teardrop shape and defining a fluid port, wherein the spigot is coupled to the luer slip and the fluid port is in fluid communication with the lumen at the second end of the luer slip, and the spigot defines at least one gripping feature.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include.” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A male luer connector, comprising:
a luer slip comprising a first end and a second end, and defining a lumen extending between the first end and the second end, wherein the first end is configured to engage with a mating connector;
a collar disposed around the luer slip, wherein the collar is translatable relative to the luer slip and the collar is configured to couple with the mating connector adjacent to the first end of the luer slip; and
a spigot defining a fluid port, wherein the spigot is coupled to the luer slip and the fluid port is in fluid communication with the lumen at the second end of the luer slip.

2. The male luer connector of claim 1, wherein the second end of the luer slip is configured to receive tubing.

3. The male luer connector of claim 2, wherein the lumen at the second end of the luer slip is configured to receive tubing.

4. The male luer connector of claim 2, wherein the luer slip defines a pocket concentric to the lumen configured to receive tubing.

5. The male luer connector of claim 2, wherein the tubing comprises micro bore tubing or small bore tubing.

6. The male luer connector of claim 1, wherein the fluid port of the spigot is configured to receive tubing.

7. The male luer connector of claim 6, wherein the tubing is disposed between the spigot and the luer slip.

8. The male luer connector of claim 6, wherein the tubing comprises standard bore thick wall tubing, standard bore tubing or macro bore tubing.

9. The male luer connector of claim 1, wherein the luer slip is formed from polypropylene.

10. The male luer connector of claim 1, wherein the collar is formed from polypropylene.

11. The male luer connector of claim 1, wherein the spigot is formed from polypropylene.

12. An infusion set, comprising:
an infusion set component;
tubing coupled to the infusion set component; and
a male luer connector, comprising:
a luer slip comprising a first end and a second end, and defining a lumen between the first end and the second end, wherein the first end is configured to engage with a mating connector, and the luer slip is coupled to the tubing.

13. The infusion set of claim 12, wherein the tubing is coupled to the second end of the luer slip.

14. The infusion set of claim 12, wherein the tubing is received in a pocket defined by the luer slip, wherein the pocket is concentric to the lumen.

15. The infusion set of claim 12, wherein the tubing comprises micro bore tubing or small bore tubing.

16. The infusion set of claim 12, further comprising a spigot defining a fluid port, wherein the spigot is coupled to the luer slip, the fluid port is in fluid communication with the lumen at the second end of the luer slip, and the tubing is coupled to the fluid port.

17. The infusion set of claim 16, wherein the tubing is disposed between the spigot and the luer slip.

18. The infusion set of claim 16, wherein the tubing comprises standard bore thick wall tubing, standard bore tubing, or macro bore tubing.

19. The infusion set of claim 12, further comprising a collar disposed around the luer slip, wherein the collar is translatable relative to the luer slip and the collar is configured to couple with the mating connector adjacent to the first end of the luer slip.

20. An infusion set luer connector assembly, comprising:
a female luer connector, comprising:
a receiving portion;
a first fluid port configured to receive intravenous tubing; and
a female luer connector lumen fluidly coupled between the receiving portion and the first fluid port; and
a male luer connector, comprising:
a luer slip comprising a first end and a second end, and a plurality of protrusions extending radially from a slip surface, and the luer slip defining a lumen extending between the first end and the second end, wherein the first end is configured to engage with a mating connector;
a collar disposed around the luer slip and defining a plurality of notches corresponding to the plurality of protrusions of the luer slip, wherein the collar is translatable relative to the luer slip, the collar is configured to couple with the mating connector adjacent to the first end of the luer slip, and the plurality of notches are configured to engage with the plurality of protrusions when the collar is spaced apart from the first end of the luer slip, selectively rotationally coupling the collar and the luer slip; and
a spigot comprising a teardrop shape and defining a fluid port, wherein the spigot is coupled to the luer slip and the fluid port is in fluid communication with the lumen at the second end of the luer slip, and the spigot defines at least one gripping feature.
